(19) United States
(12) Patent Application Publication
(10) Pub. No.: US 2003/0204258 A1
(43) Pub. Date: Oct. 30, 2003

(54) Posterior chamber phakic lens

(76) Inventors: William M. Graham, Fort Worth, TX (US); Stephen J. Van Noy, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/131,333

(22) Filed: Apr. 24, 2002

(51) Int. Cl. A61F 2/16

(52) U.S. Cl. 623/6.47; 623/6.49; 623/6.51

(57) ABSTRACT

A posterior chamber phakic lens made from a foldable, highly biocompatible material. The lens has a generally circular optic and integrally formed, closed-loop haptics. The haptics project posteriorly from the optic and have generally scalloped outer edges when viewed in plan view. Such a construction helps to avoid pupillary blockage and allows for improved aqueous flow around the natural lens.

### BACKGROUND OF THE INVENTION

This invention relates generally to the field of intraocular lenses (IOL) and, more particularly, to posterior chamber phakic IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Barraquer investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). Recently, the use of photablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. All of these refractive surgical procedures cause an irreversible modification to the shape of the cornea in order to effect refractive changes, and if the correct refraction is not achieved by the first procedure, a second procedure or enhancement must be performed. Additionally, the long-term stability of the correction is somewhat variable because of the variability of the biological wound healing response between patients.

Several companies are investigating implantable posterior chamber phakic IOLs, including the Staar ICL lens and the Medennium PRL lens. These and other anterior chamber phakic lenses are described in U.S. Pat. No. 4,769,035 (Kelman), U.S. Pat. No. 6,015,435 (Valunin, et al.) and U.S. Pat. No. 6,106,553 (Feingold), the entire contents of which being incorporated herein by reference. The clinic experience with commercially available anterior chamber phakic lenses has not been entirely satisfactory due to pupillary block, unwanted rotation of the lens and the development of traumatic cataract.

Therefore, a need continues to exist for a safe, stable and biocompatible posterior chamber phakic intraocular lens.

### BRIEF SUMMARY OF THE INVENTION

The present invention improves upon the prior art by providing a posterior chamber phakic lens made from a foldable, highly biocompatible material. The lens has a generally circular optic and integrally formed, closed-loop haptics. The haptics project posteriorly from the optic and have generally scalloped outer edges when viewed in plan view. Such a construction helps to avoid pupillary blockage and allows for improved aqueous flow around the natural lens.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible intraocular lens that allows for increased aqueous flow to the natural lens.

These and other advantages and objectives of the present invention will become apparent from the detailed description and claims that follow.

### BRIEF DESCRIPTION OF THE DRAWING

FIG. 1 is an enlarged top plan view of the lens of the present invention.

FIG. 2 is an enlarged cross-sectional view of the lens of the present invention taken at line 2-2 in FIG. 1.

FIG. 3 is an enlarged side view of the lens of the present invention.

### DETAILED DESCRIPTION OF THE INVENTION

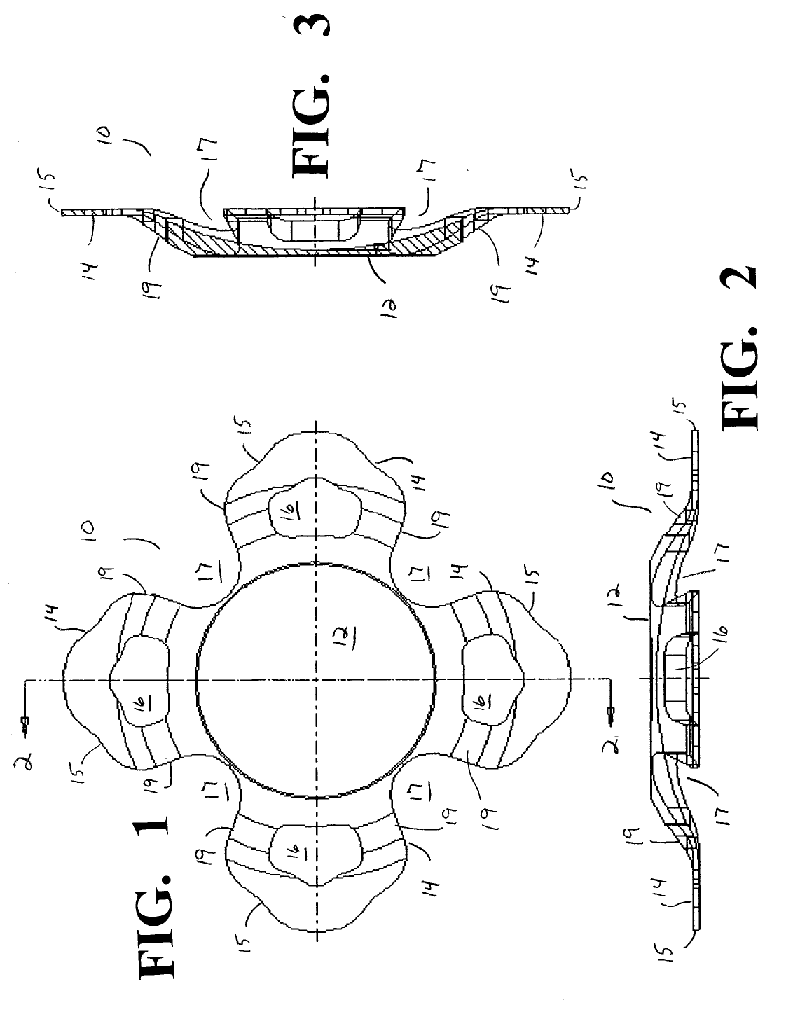

As best seen in FIGS. 1, 2 and 3, lens 10 of the present invention generally includes optic 12 and at least two closed loop haptics 14 integrally formed with optic 12. Optic 12 may be of any suitable size, such as between 4.5 mm and 6.5 mm in diameter, and may be biconcave, biconvex, concave/convex or any other suitable geometry. Optic 12 may also contain refractive or diffractive features, such features being well-known in the art. Lens 10 is preferably formed in any suitable overall diameter, for example, around 12 millimeters, for implantation in the posterior chamber in front of the natural lens from a soft, foldable material such as a hydrogel, silicone or soft acrylic, such diameters and materials being well-known in the art. As best seen in FIGS. 2 and 3, haptics 14 may project or vault posteriorly from optic 12, so as to locate optic 12 anteriorly of haptics 14 once implanted in an eye. One skilled in the art will recognize, however, that lens 10 may also be planar. Haptics 14 contain central opening 16. Outer periphery 15 of haptics 14 have a generally scalloped shape and haptics 14 are separated by notches 17. Although FIG. 1 illustrates openings 16 as being oval, one skilled in the art will recognize that openings 16 may be round, oval or any other suitable shape and oriented in any suitable direction. The vaulting of optic 12 anteriorly, along with openings 16 and the scalloped shape of the outer periphery of haptics 14, allow for increased aqueous flow around the natural lens and reducing pupillary blockage. In addition, grooves 19 in haptics 14 extend between notches 17 and openings 16 and also provide increased aqueous flow around the natural lens.

This description is given for purposes of illustration and explanation. It will be apparent to those skilled in the relevant art that changes and modifications may be made to the invention described above without departing from its scope or spirit.

We claim:

1. An intraocular lens, comprising:
a) an optic; and
b) at least two closed loop haptics connected to the optic, the haptics containing an opening and having a generally scalloped shaped outer periphery.

2. The lens of claim 1 wherein the haptics are integrally formed with the optic.

3. The lens of claim 1 wherein the optic comprises a soft acrylic.

4. The lens of claim 1 wherein the optic comprises a hydrogel.

5. The lens of claim 2 wherein the lens comprises a soft acrylic.

6. The lens of claim 2 wherein the lens comprises a hydrogel.

7. The lens of claim 1 wherein the optic comprises silicone.

8. The lens of claim 2 wherein the lens comprises silicone.

9. The lens of claim 1 further comprising at least one notch separating the haptics.

10. The lens of claim 9 further comprising a groove between the notch and the opening.

11. An intraocular lens, comprising:
a) an optic; and
b) at least two closed loop haptics connected to the optic and separated by a notch, the haptics having a generally scalloped shaped outer periphery, an opening and a groove that allows for aqueous flow between the notch and the opening.

12. The lens of claim 11 wherein the haptics are integrally formed with the optic.

13. The lens of claim 11 wherein the optic comprises a soft acrylic.

14. The lens of claim 11 wherein the optic comprises a hydrogel.

15. The lens of claim 12 wherein the lens comprises a soft acrylic.

16. The lens of claim 12 wherein the lens comprises a hydrogel.

17. The lens of claim 11 wherein the optic comprises silicone.

18. The lens of claim 12 wherein the lens comprises silicone.
